(19) United States
(12) Patent Application Publication
(10) Pub. No.: US 2020/0390595 A1
(43) Pub. Date: Dec. 17, 2020

(54) PATIENT WARMING APPARATUS

(71) Applicant: Keystone Manufacturing, LLC d/b/a Keystone Solutions Group, Kalamazoo, MI (US)

(72) Inventors: James Thomas Gaffney, Hinsdale, IL (US); James Arthur Medsker, Lawton, MI (US); Vaughn Gerber, Portage, MI (US); Andrew William Verstraete, Kalamazoo, MI (US)

(21) Appl. No.: 16/904,483

(22) Filed: Jun. 17, 2020

Related U.S. Application Data
(60) Provisional application No. 62/862,669, filed on Jun. 17, 2019.
(60) Provisional application No. 62/931,638, filed on Nov. 6, 2019.
(60) Provisional application No. 62/994,110, filed on Mar. 24, 2020.

(51) Int. Cl. A61F 7/02; A61F 7/00

(52) U.S. Cl. CPC A61F 7/02; A61F 7/007; A61F 2007/0095; A61F 2007/0093; A61F 2007/0039; A61F 2007/0247; A61F 2007/0078; A61F 2007/0029; A61F 2007/0094

(57) ABSTRACT

A patient warming apparatus having a central assembly and optionally extremity assemblies. The central assembly includes a heating body. The heating body comprises an inner heating fabric and an outer covering. The central assembly further includes a control module, a temperature sensor both attachable to a power supply. The extremity assemblies are attachable to the control module of the central assembly.

### CROSS-REFERENCE TO RELATED APPLICATION

This application claims priority from each one of U.S. Patent Application Ser. No. 62/994,110, filed Mar. 24, 2020, entitled “Patient Warming Apparatus”, U.S. Patent Application Ser. No. 62/862,669 filed Jun. 17, 2019, entitled “Patient Warming Apparatus” and U.S. Patent Application Ser. No. 62/931,638 filed Nov. 6, 2019, entitled “Patient Warming Apparatus”. The entire specification of each of the three foregoing priority patent applications is incorporated by reference in their respective entirety into the present disclosure.

### BACKGROUND OF THE DISCLOSURE

### 1. Field of the Disclosure

The disclosure relates in general to medical devices, and more particularly, to a patient warming apparatus, that can be utilized during surgical procedures, and throughout the continuum of care for a patient (i.e., pre-op, transportation, post-op, recovery, infusion procedures (dialysis, chemo), ambulance, rescue, military, among others). It will be understood that the patient warming apparatus can be utilized at times other than during surgical procedures, and the reference to surgical procedures is purely exemplary and not to be deemed limiting.

### 2. Background Art

It is known that during surgery and other procedures, it is often the case that a patient's body may have difficulty maintaining normal ranges of body temperature. In many instances, the core body temperature can be reduced to a point where it is below the normal range for humans (typically below 98.6° F.). while some reduction in core body temperature can be expected, problems arise when the temperature of the patient falls below these lower thresholds.

For example, if a patient experiences hypothermia, studies have shown that surgical site infections increase three-fold. The reduction in temperature directly impairs immune function and can make a patient susceptible to infections during and after a procedure. Bleeding also increases, thereby increasing the need for blood transfusions. The patient in hypothermia experiences a decrease in the body's natural ability to form clots to stop bleeding. Additionally, healing is delayed.

Events such as hypothermia and its effects increase an average hospital stay by almost ten extra days, increasing the cost to the patient. Additionally, hospitals experience a greatly increased cost of operation due to the complications caused directly by hypothermia. For this reason it is estimated that more than two and a half billion dollars are currently spent in the US alone on patient warming.

Some devices have been developed to provide patient warming to avoid hypothermia. One such device is the Bair Hugger offered by 3M Corporation of Minneapolis, Minn. Another is the Hot Dog offered, both of which are incorporated by reference herein. These solutions suffer from multiple drawbacks. First, the solutions require a substantial power draw, in excess of 800 W and as much as 1500 W. Furthermore, some of the devices are difficult to set up, they increase the risk of air borne contaminants that lead to increased infection. In addition, these solutions can lead to localized hot zones which can lead to patient burns.

### SUMMARY OF THE DISCLOSURE

The disclosure is directed to a patient warming assembly having a central assembly and optionally extremity assemblies. The patient warming assembly is structurally configured to be a relatively thin configuration which can follow the contours of the body. The central assembly can be positioned over or under to torso of a patient. The extremity assemblies comprise an over-extremity wrap which can be placed over an extremity such as an arm or a leg.

It is envisioned that the central assembly and the extremity assembly are constructed from biocompatible materials, that they are highly flexible and conformable to the body of the patient. It is contemplated that the outer perimeter may include weights (often termed edge-weighted) to warp around the body or extremities, toward maximizing contact surface area.

It is contemplated that the apparatus is of relatively low power consumption, and it is contemplated that a DC power source can be utilized to power the device, which may be adapted from a wall outlet or which may be obtained from a battery or the like.

In another aspect of the disclosure, the disclosure is directed to a patient warming apparatus comprising a central assembly. The central assembly comprises an outer covering and an inner heating fabric. The outer covering has a top sheet and a bottom sheet defining a perimeter and a volume therebetween. The inner heating fabric has a first side and a second side. A first side bus bar is coupled to the first side and a second side bus bar is coupled to the second side. The inner heating fabric is positioned within the outer covering. A control module is electrically coupled to the first side bus bar and the second side bus bar. A temperature sensor coupled to the control module and positioned within the perimeter of the outer covering.

In some configurations, the inner heating fabric comprises a plurality of conductive filaments within a woven matrix. The plurality of conductive filaments extend from the first side bus bar and the second side bus bar and electrically coupled thereto.

In some configurations, the plurality of conductive filaments comprise a plurality of sinusoidal filaments that extend between the first side bus bar and the second side bus bar spaced apart from each other, and generally parallel to each other.

In some configurations, the first side bus bar and the second side bus bar each comprise a plurality of layers of conductive fabric. The plurality of layers of conductive fabric are stitched to the inner heating fabric so as to place the conductive filaments into electrical communication with the conductive fabric.

In some configurations, the conductive fabric of the first side bus bar overlies a first side edge of the inner heating fabric. Additionally, the conductive fabric of the second side bus bar overlies a second side edge of the inner heating fabric.

In some configurations, the first side bus bar and the second side bus bar are positioned substantially parallel to each other.

In some configurations, the apparatus may further comprise an extremity assembly electrically coupled to and extending from the central assembly. The extremity assembly structurally configured to be wrapped around an extremity of a patient. The extremity assembly comprises an extremity top sheet and an extremity bottom sheet defining a volume with an extremity inner heating fabric positioned therewithin.

In some configurations, the extremity inner heating fabric further comprises a plurality of extremity conductive filaments positioned in an extremity matrix, with a first side extremity bus bar and a second side extremity bus bar electrically coupled to opposing sides thereof. The bus bars are coupled to the control module.

In some configurations, the extremity top and bottom sheets include a perimeter and the inner heating fabric further includes an inner heating fabric perimeter. The extremity top and bottom sheets extend beyond the inner heating fabric perimeter so as to facilitate the folding of the extremity top and bottom sheets over the inner heating fabric, to, in turn, wrap an extremity of a patient therewithin.

In some configurations, the inner heating fabric is substantially rectangular, with the top and bottom sheets being substantially triangular or polygonal.

In some configurations, the extremity assembly is coupled to the central assembly through a magnetic coupling, and electrically through an inductive coupling which is engageable upon joining through the magnetic coupling.

In some configurations, a plurality of extremity assemblies are coupled to the central assembly.

In some configurations, the extremity assembly includes at least one temperature sensor in communication with the control module.

In some configurations, the central assembly is structurally configured to extend over a torso of a patient. The apparatus further comprises a plurality of extremity assemblies, at least one of which is structurally configured to extend over a first leg of a patient, a second leg of a patient, a first arm of a patient and a second arm of a patient.

In some configurations, the central assembly further includes a plurality of extremity attachment ports structurally configured to receive an extremity coupling jack of an extremity assembly.

In some configurations, the central assembly has an operating wattage that is less than 800 W, and more preferably less than 400 W, and more preferably less than 200 W and more preferably less than 100 W and more preferably approximately 50 W.

In some configurations, the patient warming apparatus is coupled to one of an electrical outlet and a battery.

In some configurations, the operating wattage is less than 115 W/m².

In some configurations, the apparatus is configured to operate with a 24 Volt system, wherein resistance of the inner heating fabric between the first side bus bar and the second side bus bar is approximately 10.8 Ohms.

### BRIEF DESCRIPTION OF THE DRAWINGS

The disclosure will now be described with reference to the drawings wherein:

FIG. 1 of the drawings is a top plan view of a patient utilizing a patient warming apparatus of the present disclosure, with the central assembly being positioned under the user;

FIG. 1a of the drawings is a top plan view of a patient utilizing a patient warming apparatus of the present disclosure, with the central assembly being positioned over the user;

FIG. 2 is a schematic representation of a taken apart central assembly of the present disclosure, separated at the seam so as to allow exposure of the inner components thereof;

FIG. 3 of the drawings is a schematic representation of the inner heating fabric of the present disclosure;

FIG. 4 of the drawing is a schematic representation of the extremity assembly of the present disclosure;

FIG. 5a through 5c of the drawings are schematic representations of the central assembly having perimeter weights, or, an extremity assembly having perimeter weights;

FIG. 5d of the drawings is a schematic representation of the central assembly having a patient positioned thereon;

FIGS. 6a through 6e of the drawings are different configurations of the central assembly and different positions and orientations thereof;

FIGS. 7a through 7d of the drawings is a collective schematic representation of the steps to wrap a hand of a patient;

FIGS. 8a through 8d of the drawings is a collective schematic representation of the steps to wrap a foot of a patient; and

FIGS. 9a through 9c of the drawings is a collective schematic representation of a rolled up or folded up central assembly positionable on a backpack or the like;

FIG. 10 of the drawings is a schematic representation of an inductive coupling between the warming apparatus and the extremity assembly;

FIG. 11 of the drawings is a perspective view of another configuration of the patient warming apparatus;

FIG. 12 of the drawings is a perspective view of an inner heating fabric prior to formation of the first and second side bus bars;

FIG. 13 of the drawings is a perspective view of the configuration of the patient warming apparatus of FIG. 12 when the bus bars are rolled;

FIG. 14 of the drawings is a perspective view of the configuration of the patient warming apparatus of FIG. 12 with the bus bars being stitched;

FIG. 15 of the drawings is a perspective view of the configuration of the patient warming apparatus of FIG. 12 with the conductor coupled to the bus bars; and

FIG. 16 of the drawings is an exploded perspective view of the configuration of the patient warming apparatus of FIG. 12 with the outer covering laminated thereto.

### DETAILED DESCRIPTION OF THE DISCLOSURE

While this disclosure is susceptible of embodiment in many different forms, there is shown in the drawings and described herein in detail a specific embodiment(s) with the understanding that the present disclosure is to be considered as an exemplification and is not intended to be limited to the embodiment(s) illustrated.

It will be understood that like or analogous elements and/or components, referred to herein, may be identified throughout the drawings by like reference characters. In addition, it will be understood that the drawings are merely schematic representations of the invention, and some of the components may have been distorted from actual scale for purposes of pictorial clarity.

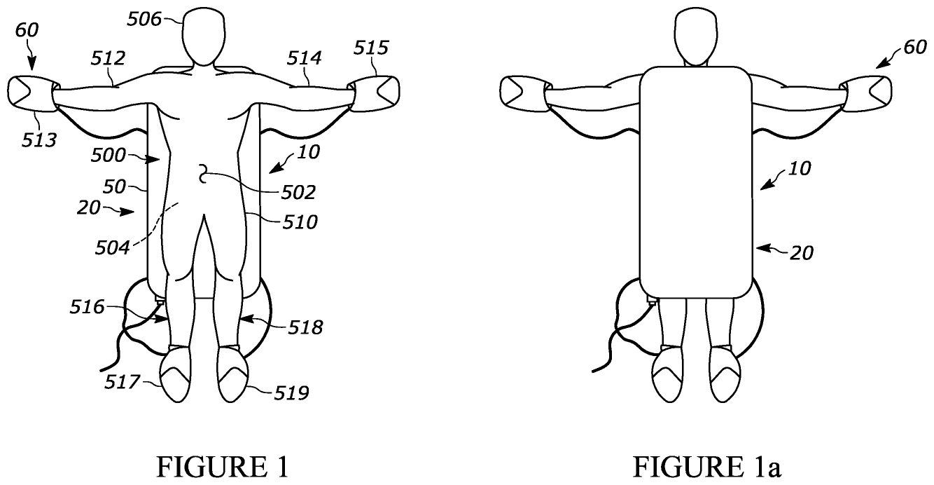

Referring now to the drawings and in particular to FIG. 1 (and also FIG. 1a), an exemplary patient heating assembly is shown at 10. It will be understood that the heating assembly shown in FIG. 1 is configured to heat a patient, such as patient 500. Patient 500 is defined by an upper surface 502 and a lower surface 504. The patient 500 has a central body or torso 510 which has a head 506 extending therefrom. A first arm 512 extends from one side and terminates at first hand 513. The second arm 514 extends from the opposite side and terminates at second hand 515. A first leg 516 extends from the torso and terminates at first foot 517. A second leg 518 extends from the torso and terminates at second foot 519. It will be understood as well that the patient warming apparatus 10 can have a number of different configurations, many of which will be discussed below.

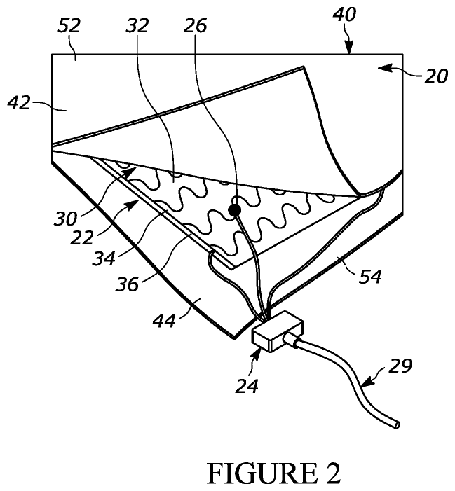

With reference to FIGS. 2 through 4, the patient warming apparatus includes a central assembly 20 and extremity assemblies, such as extremity assembly 60. In the configuration shown, the central assembly corresponds to the portion of the patient that includes the torso and portions of the legs. In some configurations, the central assembly can be configured to be positioned below the patient so as to contact the lower surface 504, whereas in other configuration, it may be positioned above the patient, as a covering. The extremity assembly 60 can be used as a wrap to wrap different extremities, such as, for example, the hands or the feet or the head.

The central assembly 20 is shown as comprising heating body 22, control module 24, temperature sensors 26, extremity attachment port 29 and power supply (not shown). The heating body 22 comprises an inner heating fabric 30 and an outer covering 40. The heating body defines a perimeter 50 and a thickness, as well as a top surface 52 and a bottom surface 54. It is contemplated that the heating body is flexible like a fabric, and has a minimal thickness. For example, it is contemplated that the thickness of the heating body is less than 20 mils, and more preferably less than 15 mils and more preferably less than 10 mils and more preferably less than 8 mils and more preferably about 5 mils. Of course, these are merely exemplary and other thicknesses are contemplated.

The inner heating fabric 30 includes a woven matrix 32, a plurality of conductive filaments 34, a first side bus bar 36 and a second side bus bar 38. The woven matrix 32 comprises a polyethylene woven fabric, while variations are contemplated. The conductive filaments 34 comprises a plurality of conductive carbon resistive conductors that are positioned in a undulating (sinusoidal or the like) pattern from a first end to a second end of the woven matrix. In the configuration shown, a plurality of spaced apart and generally parallel undulating conductors are shown. A first side bus bar 36 can be sewn to the fabric and the conductive filaments at a first side to join the first ends thereof together. A second side bus bar 38 can be sewn to the fabric and the conductive filaments on a second side to join the second ends thereof together. In the configuration shown, the first side bus bar and the second side bus bar are substantially parallel to each other.

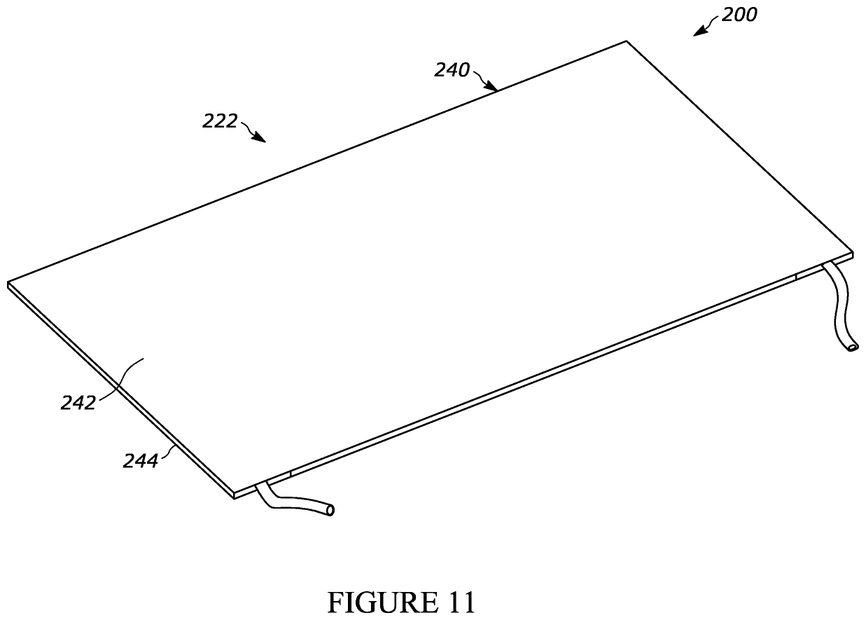

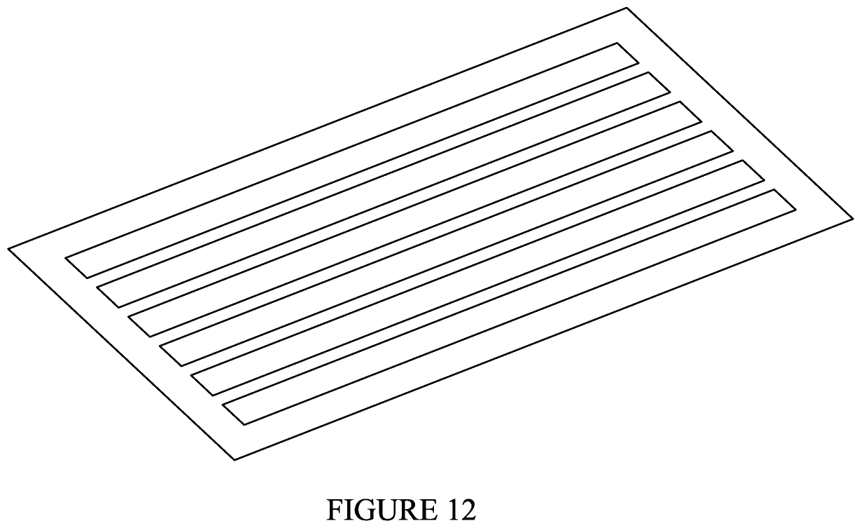

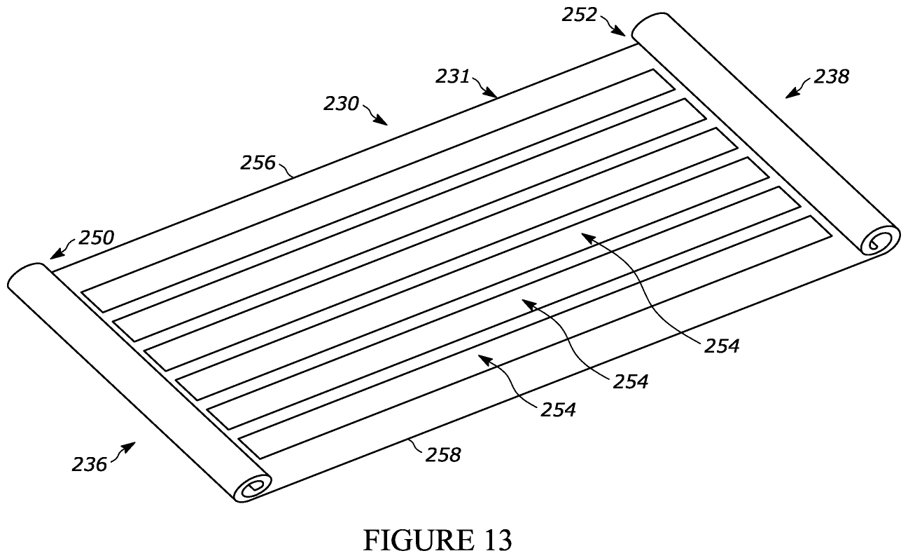

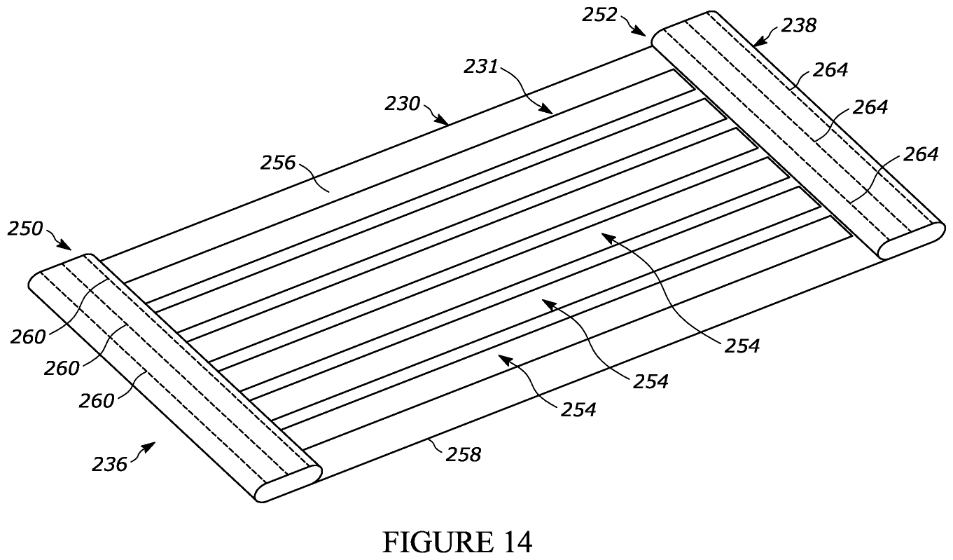

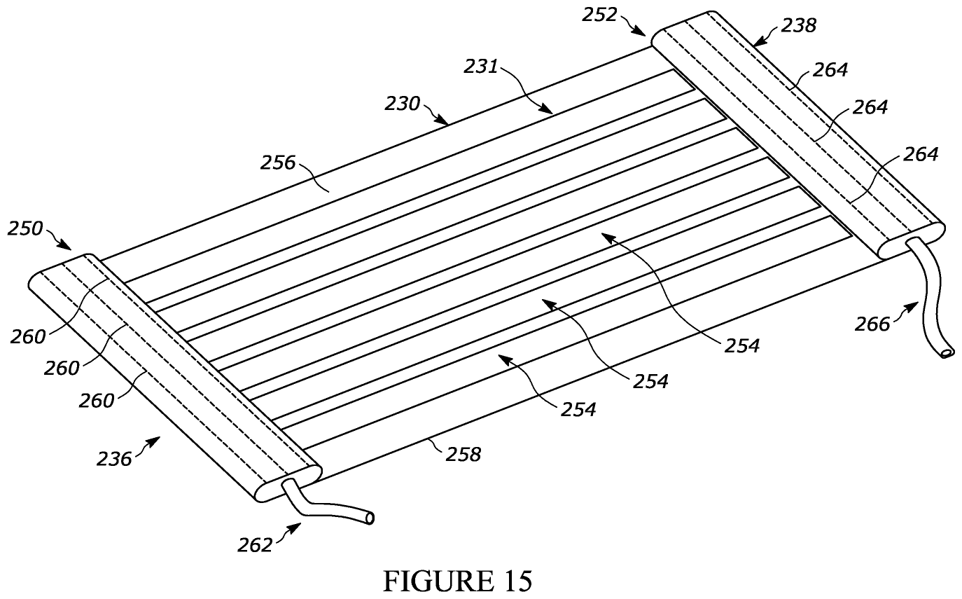

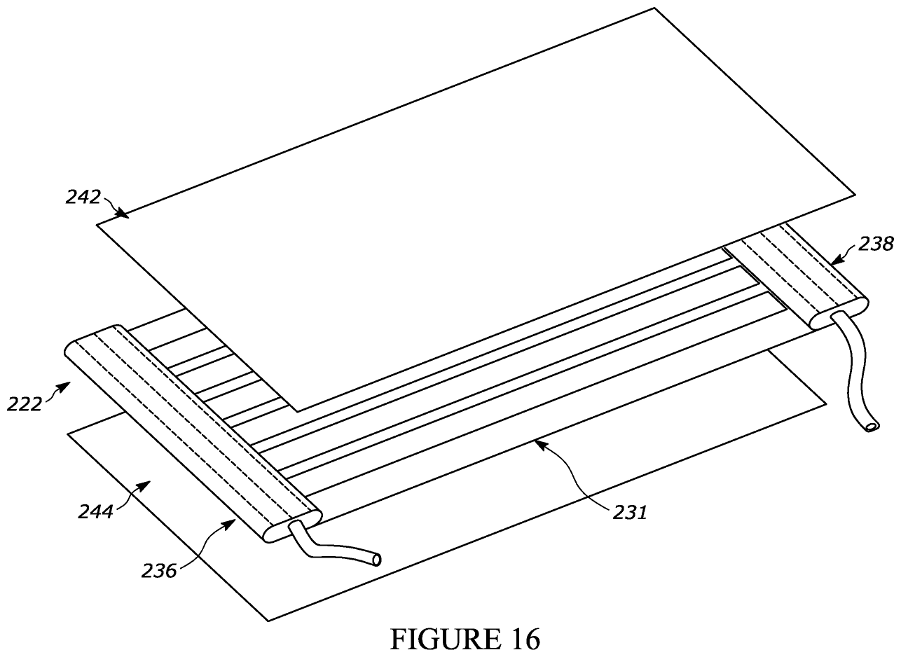

The bus bars in some configurations may comprise a woven conductive fabric 230 of FIGS. 11 through 16. For example, and among other configurations, fabric such as the woven conductive fabric may be folded over into multiple plies (similar to that which is shown in the FIGS. 11 through 16 and stitched (with conductive or non-conductive thread or the like) to the sides of the woven matrix so as to be in communication with the conductive filaments 34 thereof. The woven matrix may be inserted between plies or may be at the bottom or top of the plies. The plies may be formed from a single material folded over itself, or, may comprise a plurality of discrete sheets that are stacked relative to each other to form plies. Alternatively, the separate sheets may be both layered and folded over each other. In other configurations, the bus bars may comprise metal foil, metal

In a configuration of the present disclosure, the woven matrix and the conductive filaments may comprise an integrally sourced fabric. For example, one such fabric comprises a textile sold by Gustav Gerster GmbH & Co. KG, of Biberach, Germany as product number 15043, the entire specifications of which are hereby incorporated by reference in their entirety. Such a material has a thickness of 0.5 mm. In a 16 cm width, the material has a weight of 53.3 g/m. It is contemplated that temperatures approaching a maximum of 180° C. can be reached (while lower temperatures are contemplated). In a one meter length, the fabric has a resistance of 11.8 ohms. The foregoing fabric is exemplary and it is contemplated that a number of different fabrics are contemplated for use, as well as a number of different conductive filaments which may be formed from various conductive materials, including carbon fibers, metal filaments and wires, among others. Sewable bus bars can also be obtained from the same supplier.

The outer covering 40 comprises a top sheet 42 and a bottom sheet 44. These sheets are coupled together at the outer perimeter of the same to form a seam 48 and so as to define volume 46. The inner heating fabric is placeable within the volume 46 formed by the outer covering. In the configuration shown, the outer covering comprises a woven polyurethane that is highly water resistant, while other materials are contemplated. It is contemplated that the outer covering may be joined through heat seals (or RF welds, or ultrasonic welding, among other solutions). In other configurations, the outer covering may be joined through adhesion, or through sewing, or through various combinations of any of the foregoing. In some configurations, the heating body may be attached to one of the top sheet or the bottom sheet, or both at the seams or along other regions thereof.

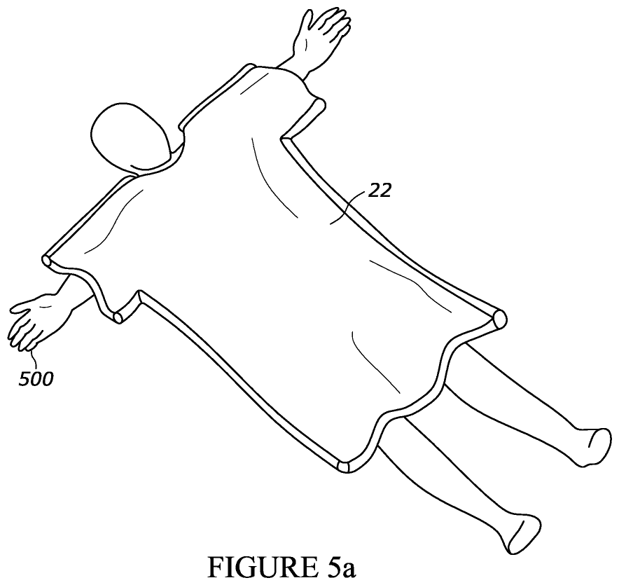

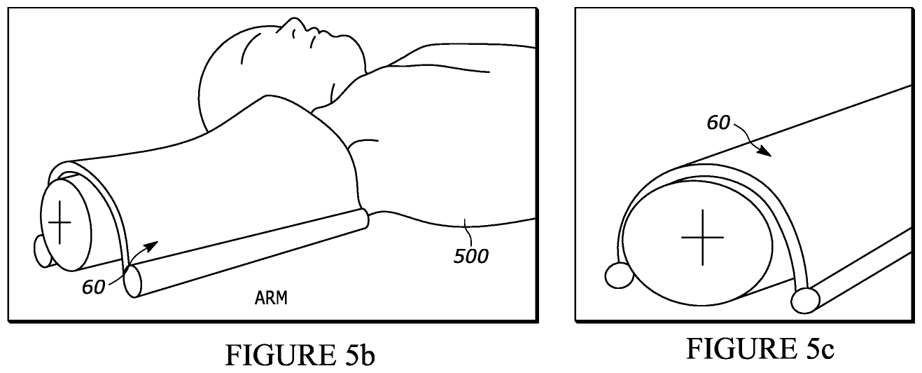

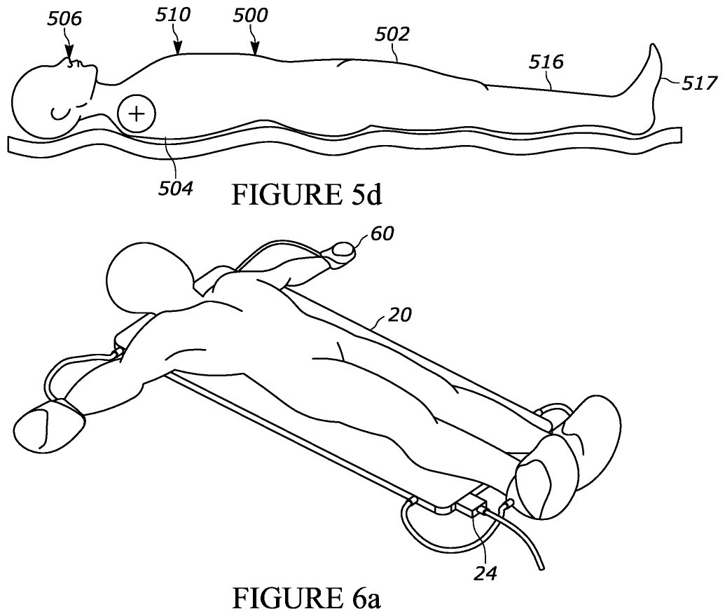

With reference to FIGS. 5a through 5c, It is contemplated that weights may be placed along the perimeter to add weight to the perimeter, or portions thereof for both the central assembly and the extremity assemblies. Such addition of weights may assist the heating body to more closely follow the contours of a patient, thereby increasing the direct contact area of the extremity heating body with the patient. As is shown in FIG. 5d, the central assembly can follow the contours of the body to maximize the direct contact therebetween.

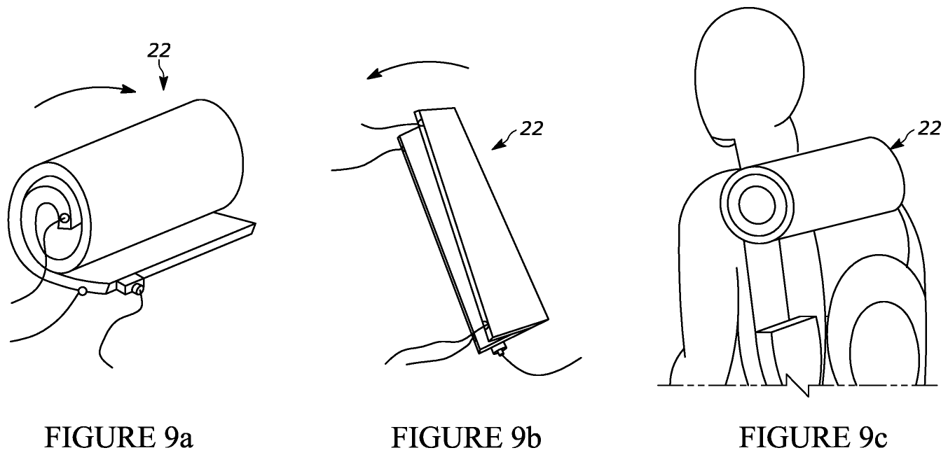

With reference to collective FIGS. 9a through 9c, it is further contemplated that the central assembly can be rolled up or folded up for storage and transport. Due to the light weight nature of the central assembly, the rolled up central assembly can be carried on, for example, a backpack or the like.

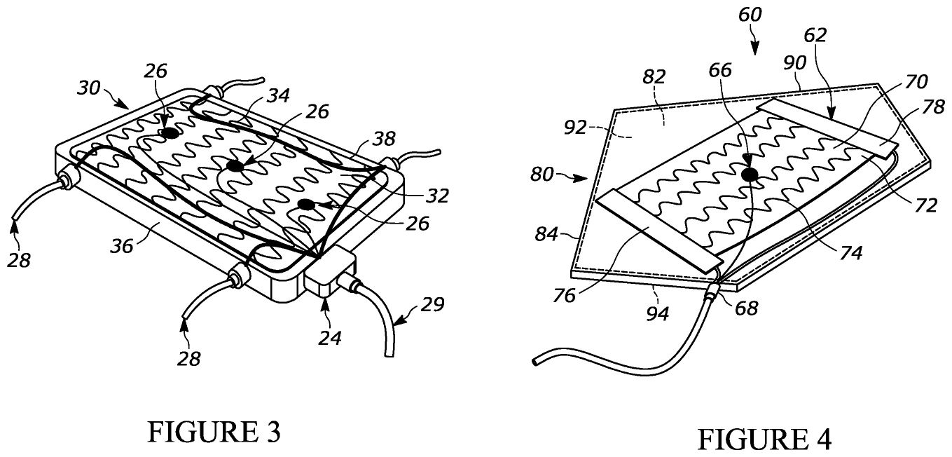

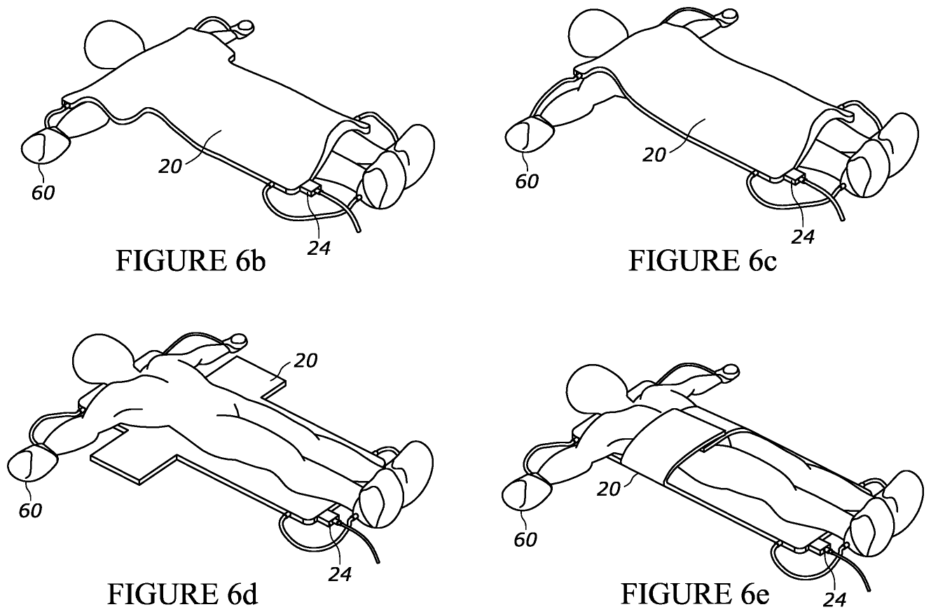

Referring again to FIGS. 2 and 3 (as well as FIGS. 6a through 6e) the control module 24 is positioned along the perimeter (and may be joined at the perimeter to the heating body, or the outer covering, or both. The control module is electrically in communication with each of the first side bus bar and the second side bus bar and controls the current through (or voltage across) the conductive filaments to achieve the desired temperature of the filaments (or conditions surrounding the filaments). A plurality of temperature sensors, such as temperature sensor 26, may be positioned along various regions of the heating body 22. It will be understood that the temperature sensors are coupled to the control module electrically so as to provide feedback to the control module. The particular location of the sensors and the type of sensors utilized can be varied, as will be understood by one of ordinary skill in the art. It is desirable to avoid hot spots or cold spots, so as to provide heat uniformly.

The central assembly is shown as having a single zone, however, it is contemplated that the bus bars may have multiple zones, and may be able to be controlled individually so that different ones of the conductive filaments may have different currents extending therethrough, or may have a different voltage drop thereacross.

The control module 24 is coupled to a power supply (not shown) but in the direction of arrow 29. In the configuration shown, the control module has a wired connection extending therefrom which would be coupled to a power supply. As it is contemplated that the operating wattage is preferably less than 800 W, and more preferably less than 400 W, and more preferably less than 200 W and more preferably less than 100 W and more preferably approximately 50 W, it is contemplated that the power supply may draw power from a wall outlet, or from a battery or a combination. Advantageously, due to the relatively low power requirements, the system may be configured to be quite portable and usable where wall outlet power is not feasible or desirable. And this can be achieved without the need for a large battery. The control module may comprise requisite circuitry to provide the necessary voltage across the first bus bar and second bus bar, and may be equipped with on/off switch as well as circuitry to receive information from temperature sensors and adjust the voltage level across the bus bars. Additionally, these may be microprocessor controlled control modules that can additionally control the coupling and heating of extremity assemblies. Among other configurations, the control module may comprise simple devices that include a plurality of user manipulated switches that may be attached via a dongle to the central assembly, or which may be incorporated into the central assembly. Furthermore, in other configurations, the control module may include microprocessors and the like, and may include data ports or the ability to transfer data through wired connections (USB, among others) or through wireless connections (Bluetooth, wifi, among others). A control module would therefore include a microprocessor, addressable memory and connectivity therebetween, along with chipsets to facilitate the communication therebetween and also to gather, store and analyze data. In some configurations, the control module may be controllable through a smartphone, a special purpose remote control, or the like.

In the configuration shown, the central assembly may include a plurality of extremity attachment ports 28 that are coupled to the control module which can accept extremity coupling jacks 68 from any number of extremity assemblies 60. It will be understood that the extremity attachment ports 28 are electrically coupled to the control module 24, and, also to the power supply coupled to the control module 24. Each of the extremity coupling jacks can be attached to a respective extremity attachment port of the central assembly.

With reference to FIGS. 1, 4 and 6a through 6e, a number of different extremity assemblies 60 are contemplated, and, as such, one extremity assembly will be described with the understanding that the extremity assemblies are generally structurally quite similar in concept, while possibly being different dimensionally or otherwise.

Referring to FIG. 4, the extremity assembly 60 include extremity heating body 62, and extremity coupling jack 68. The extremity heating body 62 includes inner heating fabric 70 and outer covering 80. The inner heating fabric 70 has a configuration that is functionally like that of the inner heating fabric 30 and includes woven matrix 72, conductive filaments 74, first side bus bar 76 and second side bus bar 78. The outer covering 80 comprises top sheet 82 and bottom sheet 84 that are joined together about a perimeter 90 to form a volume. The outer covering defines a top surface 92 and a bottom surface 94. The extremity coupling jack 68 extends from the perimeter and is electrically coupled to the first and second side bus bars. In addition, temperature sensors, such as temperature sensor 66, may be positioned at desired locations along the inner heating fabric and electrically coupled to the extremity coupling jack for electrical coupling to the control module.

In the configuration shown in FIG. 1 (as well as in FIGS. 6a through 6e), it is contemplated that the extremity assemblies are configured to be utilized at each of the extremities defined by first hand 513, second hand 515, first foot 517 and second foot 519. In other configurations, fewer or greater numbers of extremity assemblies may be utilized. It is contemplated that in certain configurations, while termed extremity assemblies, such assemblies may be utilized with other parts of the body that would not be defined as extremities. That is, the particular location of use of any of the extremity assemblies is not to be deemed limited to an extremity, but use in association with extremities is contemplated.

As with the central assembly, it is contemplated that the extremity heating body has similar thicknesses and similar flexibility.

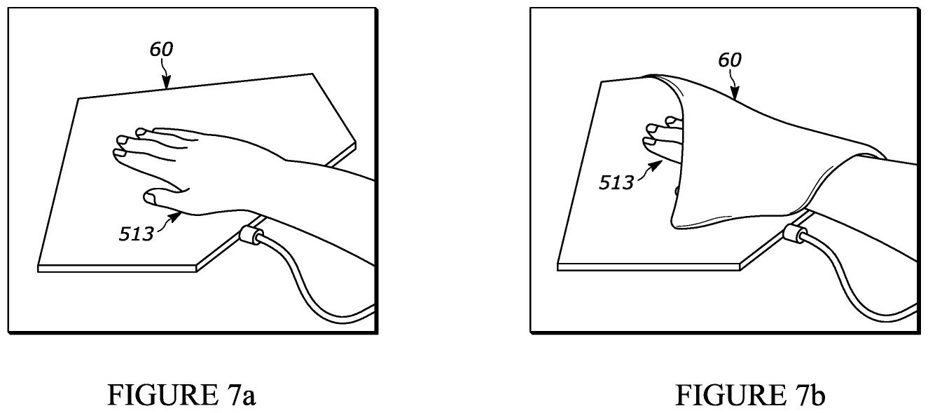

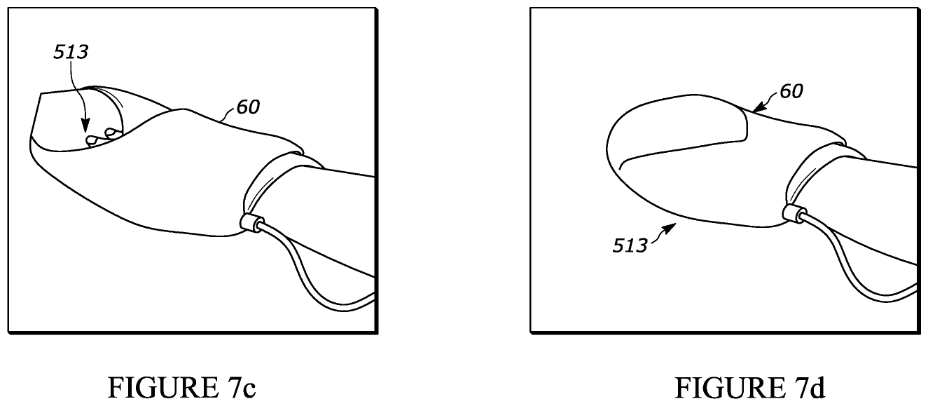

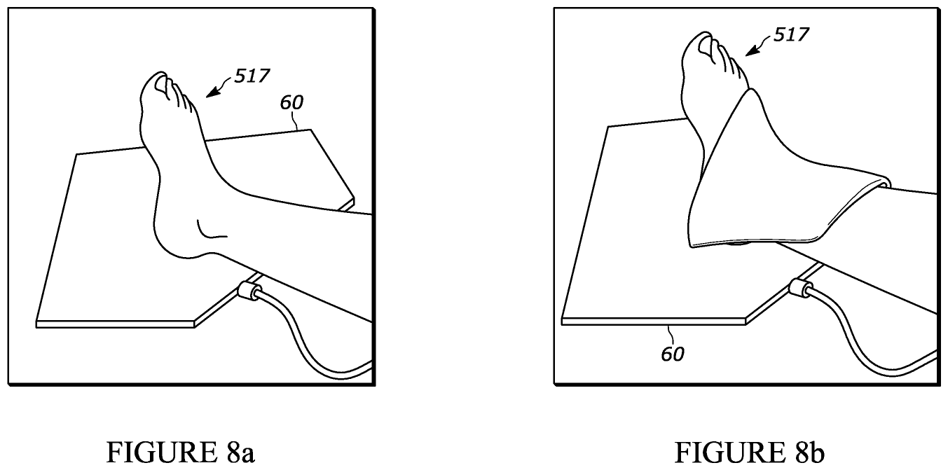

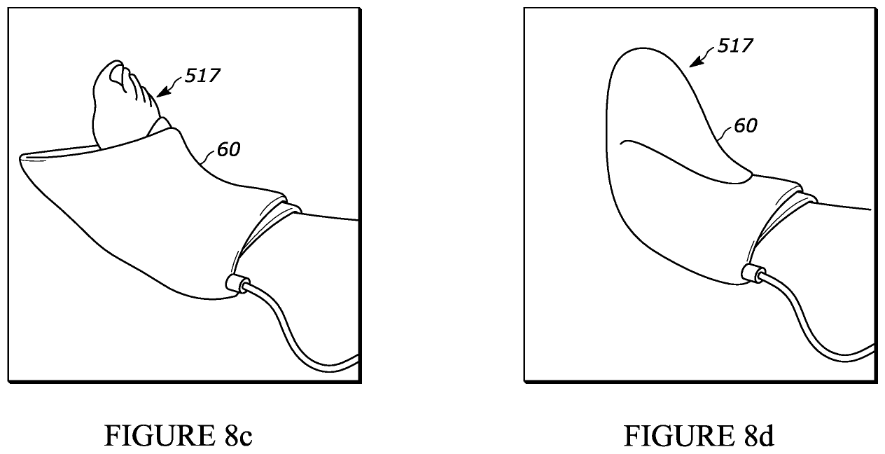

It is contemplated that the extremity heating body is substantially larger than the extremity so that it is possible to wrap the extremity with the extremity heating body. For example, and with reference to collective FIGS. 7a through 7d for a hand, and collective FIGS. 8a through 8d for a foot, the extremity of the patient is placed onto a central region of an extremity assembly. The extremity assembly is wrapped over the extremity. In the configuration shown, three separate wings are wrapped over the extremity and/or over other portions of the extremity assembly so as to envelope the extremity. Due to the flexibility of the extremity assembly, such wrapping results in intimate contact of the extremity assembly (and in particular the extremity heating body with the extremity). The configuration of the extremity assemblies allows for a substantially lie flat configuration, instead of cavities, to facilitate cleaning and sanitizing of the assemblies when not in use. To the contrary, where a cavity such as a mitten or the like is utilized (and such a shape may be utilized in association with the present disclosure), it tends to be difficult to clean adequately, thereby fostering infection.

It is contemplated that the central assembly may have a number of different configurations. For example, in FIG. 6a, the central assembly is positioned below a patient so that the patient's lower surface is in contact with the top surface of the central assembly. The central assembly comprises an elongated rectangular configuration A plurality of extremity assemblies is likewise provided for each of the hands and feet. In FIG. 6b, the central assembly comprises a T-formation that includes wings corresponding to the upper arms. In the configuration shown, the central assembly is positioned so as to lie over the upper surface of the patient. Again, extremity assemblies are provided for the hands and feet. In FIG. 6c, the central assembly is positioned as it is in FIG. 6b, with a rectangular configuration. In FIGS. 6d and 6e, the position of the central assembly is like that which is shown in FIG. 5a. The shape of the central assembly comprises a cross configuration, with a pair of opposing wings. These opposing wings can be wrapped around the torso of the user. In the configurations shown, the two opposing wings overlap each other on the upper surface of the patient. A number of different configurations are contemplated, including different shaped configurations, and configurations which may include a greater number or a fewer number of extremity assemblies.

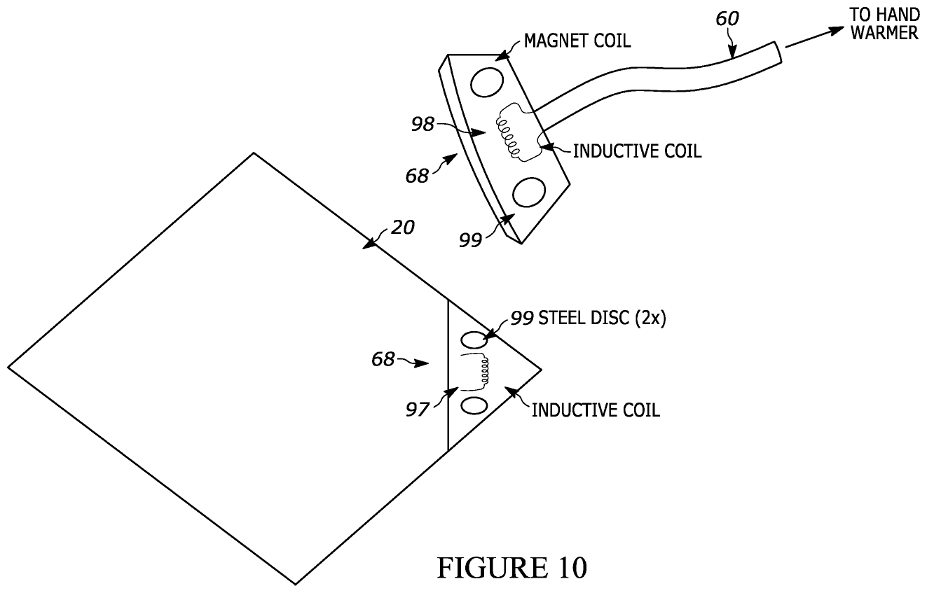

While a number of different manners of coupling of the extremity assemblies are contemplated, one such configuration is shown in FIG. 10. For example, an inductive coil 97 can be utilized to provide the electrical coupling between the extremity assemblies and the central assembly. Additionally, a magnetic system 99 can be utilized to maintain the coupling and the proper position of the two inductive coils 97, 98. As such, each can be water proof and can provide the necessary electrical circuit without requiring any plug-in style couplings. For example, magnets can be positioned on one of the coupling members proximate the inductive coil with matching ferromagnetic discs associated with the other inductive coil. Thus, the mating of the magnets with the underlying ferromagnetic discs will join the two structures and also properly align the inductive coil of the central assembly and of the extremity assembly.

Another configuration of the patient warming apparatus is shown in FIGS. 11 through 16 generally at 200. The patient warming apparatus in such a configuration includes heating body 222 which includes inner heating fabric 230 and outer covering 240. It will be understood that the same system of extremity assemblies, including those that utilize the inductive coil system shown in FIG. 10 can be incorporated into the heating body 222 of FIGS. 11 through 16. Additionally, each of the extremity systems can be modified to have the inner heating fabric of the type shown in FIGS. 11 through 16, and can utilize the construction thereof.

Referring now to FIGS. 12 through 16, the inner heating fabric 230 includes central heating portion 231, first side bus bar 236, and second side bus bar 238. It will be understood that, preferably, the entire structure of the inner heating fabric is formed from a single woven piece of conductive fabric. In some configurations, among others, and for illustrative purposes only, the following conductive fabrics are considered for use: Circuitex fabrics available under part numbers 54256-S, 5101159, 5240560, 53951348, 54209860, 5161058, 5560553, 9081460, 53951255AC, 53951255, available from Noble Biomaterials, Inc. of Scranton, Pa. The foregoing are illustrative and not to be deemed limiting, and the properties of each are hereby incorporated by reference in their entirety.

Of course, others are contemplated as well. Furthermore, it is contemplated that other fabrics may likewise be utilized to form the inner heating fabric. It is further contemplated that the inner heating fabric may be divided into a number of different assemblies to form the heating body, while a single one is shown in the disclosure.

In the configuration shown, the central heating portion includes first side 250 and second side 252, as well as top 256 and bottom 258. A plurality of voids, such as voids 254 are cut into the fabric so as to define alternative strips and voids. It will be understood that while the voids are shown as having a generally elongated rectangular configuration, a number of other configurations are likewise contemplated, such as non-uniform voids, having sinusoidal, square wave, arbitrary or interconnected strips or voids is likewise contemplated. That is, it is desirable to have a power dissipation (primarily through heat) across the central heating portion of approximately 53-55 W (preferably less than 115 W/m². For example, with a 24 Volt system, the resistance of the inner heating fabric is desirably approximately 10.8 Ohms, while other resistances and the like are contemplated.

The first side bus bar 236 comprises a rolled up portion of the fabric that is along the first side 250 of the central heating portion. The rolled up portion of the fabric includes stitching 260 that extends between the top and bottom so as to provide a close abutment between the rolled up portions, so as to promote conductivity and to minimize resistance within the first side bus bar. In the configuration shown, a total of three separate and generally parallel stitches extend along the first side bus bar 236, while the configuration is not limited thereto. The thread may be either one of conductive or non-conductive. Additionally, a conductor 262 may be stitched, riveted or otherwise joined to the first side bus bar so as to be electrically in communication therewith.

The second side bus bar 238 comprises a rolled up portion of the fabric that is along the second side 252 of the central heating portion. The rolled up portion of the fabric is a general mirror image of that of the first side bus bar 236. Similarly, the second side bus bar 238 includes stitching 264 and conductor 266.

It will be understood that the conductors 262, 266 complete the circuit across the central heating portion. It will be understood that the configuration may be altered without departing from the scope of the disclosure.

The outer covering 240, as is shown in FIG. 11, includes top sheet 242, and bottom sheet 244. The top sheet 242 and the bottom sheet 244 encase the central heating portion. In the configuration shown, the top and bottom sheets may be laminated to the inner heating fabric so as to maintain the integrity of the voids and the positioning of the portions of the central heating portion and the voids. It is contemplated that the sheets may comprise a flexible adhesive backed polymer, such as polyurethane or urethane (among other formulations). It will be understood that the laminate also precludes rucking induced shorting of the central heating portion. The laminate can be placed within a pillow case (such as a Herculite cover) and can be ultrasonically (or otherwise) coupled or welded to a flange around the inner perimeter of the cover.

The foregoing description merely explains and illustrates the disclosure and the disclosure is not limited thereto except insofar as the appended claims are so limited, as those skilled in the art who have the disclosure before them will be able to make modifications without departing from the scope of the disclosure.

What is claimed is:

1. A patient warming apparatus comprising:
a central assembly comprising:
an outer covering having a top sheet and a bottom sheet defining a perimeter and a volume therebetween;
an inner heating fabric having a first side and a second side, with a first side bus bar coupled to the first side and a second side bus bar coupled to the second side, the inner heating fabric positioned within the outer covering;
a control module electrically coupled to the first side bus bar and the second side bus bar; and
a temperature sensor coupled to the control module and positioned within the perimeter of the outer covering.

2. The patient warming apparatus of claim 1, wherein the inner heating fabric comprises a plurality of conductive filaments within a woven matrix, the plurality of conductive filaments extend from the first side bus bar and the second side bus bar and electrically coupled thereto.

3. The patient warming apparatus of claim 2 wherein the plurality of conductive filaments comprise a plurality of sinusoidal filaments that extend between the first side bus bar and the second side bus bar spaced apart from each other, and generally parallel to each other.

4. The patient warming apparatus of claim 2 wherein the first side bus bar and the second side bus bar each comprise a plurality of layers of conductive fabric, wherein the plurality of layers of conductive fabric are stitched to the inner heating fabric so as to place the conductive filaments into electrical communication with the conductive fabric.

5. The patient warming apparatus of claim 4 wherein the conductive fabric of the first side bus bar overlies a first side edge of the inner heating fabric and wherein the conductive fabric of the second side bus bar overlies a second side edge of the inner heating fabric.

6. The patient warming apparatus of claim 5 wherein the first side bus bar and the second side bus bar are positioned substantially parallel to each other.

7. The patient warming apparatus of claim 1 further comprising an extremity assembly electrically coupled to and extending from the central assembly, the extremity assembly structurally configured to be wrapped around an extremity of a patient, the extremity assembly comprises:
a extremity top sheet and an extremity bottom sheet defining a volume with an extremity inner heating fabric positioned therewithin.

8. The patient warming apparatus of claim 7 wherein the extremity inner heating fabric further comprises a plurality of extremity conductive filaments positioned in an extremity matrix, with a first side extremity bus bar and a second side extremity bus bar electrically coupled to opposing sides thereof, with the bus bars being coupled to the control module.

9. The patient warming apparatus of claim 7 wherein the extremity top and bottom sheets include a perimeter and the inner heating fabric further includes an inner heating fabric perimeter, with the extremity top and bottom sheets extending beyond the inner heating fabric perimeter so as to facilitate the folding of the extremity top and bottom sheets over the inner heating fabric, to, in turn, wrap an extremity of a patient therewithin.

10. The patient warming apparatus of claim 9 wherein the inner heating fabric is substantially rectangular, with the top and bottom sheets being substantially triangular or polygonal.

11. The patient warming apparatus of claim 7 wherein the extremity assembly is coupled to the central assembly through a magnetic coupling, and electrically through an inductive coupling which is engageable upon joining through the magnetic coupling.

12. The patient warming apparatus of claim 7 wherein a plurality of extremity assemblies are coupled to the central assembly.

13. The patient warming apparatus of claim 7 wherein the extremity assembly includes at least one temperature sensor in communication with the control module.

14. The patient warming apparatus of claim 7 wherein the central assembly is structurally configured to extend over a torso of a patient, and further comprising a plurality of extremity assemblies, at least one of which is structurally configured to extend over a first leg of a patient, a second leg of a patient, a first arm of a patient and a second arm of a patient.

15. The patient warming apparatus of claim 1 wherein the central assembly further includes a plurality of extremity attachment ports structurally configured to receive an extremity coupling jack of an extremity assembly.

16. The patient warming apparatus of claim 1 wherein the central assembly has an operating wattage that is less than 800 W, and more preferably less than 400 W, and more preferably less than 200 W and more preferably less than 100 W and more preferably approximately 50 W.

17. The patient warming apparatus of claim 1 wherein the patient warming apparatus is coupled to one of an electrical outlet and a battery.

18. The patient warming apparatus of claim 1 wherein operating wattage is less than 115 W/m².

19. The patient warming apparatus of claim 1 wherein the apparatus is configured to operate with a 24 Volt system, wherein resistance of the inner heating fabric between the first side bus bar and the second side bus bar is approximately 10.8 Ohms.
